SEQUENCE LISTING (24 sequences)

SEQ ID NO: 1 - DNA, 1695 bp, unknown
  Thermococcus sp. strain 9 degrees North
atgggcgatatgaggtactccgagctggccgaactctaccggaggcttgagaagaccacgctcaaaacgctcaagaccaagttcgtcgcggacttcctcaagaaaacacccgacgatttgctcgagatagttccctacctgattctcggcaaggtctttccggactgggacgagcgcgagctcggcgtcggcgagaagcttctcataagggccgtttcgatggcaaccggcgtccccgagagggaaatcgagaactcgattaaggacaccggcgatttgggcgagagcgtggctctggctctaaagaagaggaagcagaagagcttcttcagccagcccctcacgataaagcgcgtctacagcaccttcgttaaggttgccgaggcgagcggagaggggagccaggacaggaagatgaagtacttagcaaacctcttcatggatgctcaacccgaggagggcaagtacatagccagaaccgtcctcgggacgatgaggacgggcgtcgccgagggaatcctgcgcgatgccatagcggagtccttcaaggtgaagccagagctcgtcgagagggcctacatgctcacgagcgacttcggctacgtggcgaaggtcgccaagctcgaggggaacgaggggctctcgaaggtcagcatacagattgggaagccgataaggccgatgctagctcaaaacgccgccaacgtcaaggaagcgctaatcgagatgggcggtgaggcggccttcgagattaagtacgacggcgcgcgcgttcaggtccaccgcgatggggacagggtgataatctactcgaggaggcttgagaacgtcacccgctcgattcctgagatagttgaggcggtaaaggcctccctgaagccctctaatgtcatagtcgagggcgagctggttgccgtcggcgagaacggtcgcccgaggcccttccagtacgtcctgaggcgctttaggaggaagtacaacatcgaggagatgattgagaagattccgctcgagctcaacctcttcgacatcctctacgttgaaggagagagcctcatcgacacgaagttcgcagagaggaggaagaagctcgaggagagcgttgaggagagcgataagataaagctcgccgaacagctcgttacgaagaaggttgaagaggccgaggagttctacaagagggcccttgagctcggccacgagggcctgatggcgaagaggctggactccatctacgagcccggaaaccgcggtaagaagtggctgaagattaagcccacgatggagaaccttgacctcgtcattatcggagccgaatggggcgagggcaggcgcgcgcacctgctcggctcgttcctcgttgcggcctacgaccccgagagcggtgagttcgtcccggtgggcaaggtcgggagcggtttcaccgatgaagatttggtcgagttcaccaagatgctcaagcccctgattgtccgtgaagagggcaagttcgtcgagattgagcccaaggtcgtcatcgaggtcacctaccaggagatacagaagagccccaagtataagagcggtttcgcgcttcgcttcccgcgctacgtggcgctgagggaagataaaagcccggaggaggctgacaccatagagagggtcgcccagctctacgagctccaggagaggttcaaggcgaagaggtaatctaga

SEQ ID NO: 2 - DNA, 1695 bp, unknown
  Thermococcus sp. strain 9 degrees North
atgggcgatatgaggtactccgagctggccgaactctaccggaggcttgagaagaccacgctcaaaacgctcaagaccaagttcgtcgcggacttcctcaagaaaacacccgacgatttgctcgagatagttccctacctgattctcggcaaggtctttccggactgggacgagcgcgagctcggcgtcggcgagaagcttctcataagggccgtttcgatggcaaccggcgtccccgagagggaaatcgagaactcgattaaggacaccggcgatttgggcgagagcgtggctctggctctaaagaagaggaagcagaagagcttcttcagccagcccctcacgataaagcgcgtctacagcaccttcgttaaggttgccgaggcgagcggagaggggagccaggacaggaagatgaagtacttagcaaacctcttcatggatgctcaacccgaggagggcaagtacatagccagaaccgtcctcgggacgatgaggacgggcgtcgccgagggaatcctgcgcgatgccatagcggagtccttcaaggtgaagccagagctcgtcgagagggcctacatgctcacgagcgacttcggctacgtggcgaaggtcgccaagctcgaggggaacgaggggctctcgaaggtcagcatacagattgggaagccgataaggccgatgctagctcaaaacgccgccaacgtcaaggaagcgctaatcgagatgggcggtgaggcggccttcgagattaagtacgacggcgcgcgcgttcaggtccaccgcgatggggacagggtgataatctactcgaggaggcttgagaacgtcacccgctcgattcctgagatagttgaggcggtaaaggcctccctgaagccctctaatgtcatagtcgagggcgagctggttgccgtcggcgagaacggtcgcccgaggcccttccagtacgtcctgaggcgctttaggaggaagtacaacatcgaggagatgattgagaagattccgctcgagctcaacctcttcgacatcctctacgttgaaggagagagcctcatcgacacgaagttcgcagagaggaggaagaagctcgaggagagcgttgaggagagcgataagataaagctcgccgaacagctcgttacgaagaaggttgaagaggccgaggagttctacaagagggcccttgagctcggccacgagggcctgatggcgaagaggctggactccatctacgagcccggaaaccgcggtaagaagtggctgaagattaagcccacgatggagaaccttgacctcgtcattatcggagccgaatggggcgagggcaggcgcgcgcacctgctcggctcgttcctcgttgcggcctacgaccccgagagcggtgagttcgtcccggtgggcaaggtcgggagcggtttcaccgatgaagatttggtcgagttcaccaagatgctcaagcccctgattgtccgtgaagagggcaagttcgtcgagattgagcccaaggtcgtcatcgaggtcacctaccaggagatacagaagagccccaagtataagagcggtttcgcgcttcgcttcccgcgctacgtggcgctgagggaagataaaagcccggaggaggctgacaccatagagagggtcgcccagctctacgagctccaggagaggttcaaggcgaagaagtaatctaga

SEQ ID NO: 3 - DNA, 1695 bp, unknown
  Thermococcus sp. strain 9 degrees North
atgggcgatatgaggtactccgagctggccgaactttacagaaggcttgagaagaccacgctcaaaacgctcaagaccaagtttgtcgcggacttcctcaagaaaactcccgacgatttgctcgagatagttccctacctgattctcggcaaggtctttccggactgggacgagcgcgagctcggcgtcggggaaaagttgctcataagggccgtttcgatggcaaccggcgtccccgagagggaaatcgagaactcgattaaggacacgggcgatttgggcgagagcgtggctctggctctaaagaagaggaagcagaagagcttcttcagccagcccctcacgataaagcgcgtctacagcaccttcgttaaggttgccgaggcgagcggagaggggagccaggwcaggaagatgaagtacttagcaaatctcttcatggatgctcaacccgaggagggcaagtacatagccagaaccgtcctcggaacgatgaggacgggcgtcgccgagggaatcctgcgcgatgccatagcggaggccttcaaggtgaagccagagctcgtcgagagggcctacatgctcacgagcgacttcggctacgtggcgaaggtcgccaagctcgaggggaacgaggggctctcgaaggtcagcatacagattgggaagccgataaggccgatgctagctcaaaacgccgccaacgtcaaggaagcgctaatcgagatgggcggtgaggcggccttcgagattaagtacgacggcgcgcgcgttcaggtccaccgcgatggggacagggtgataatctactcgaggaggcttgagaacgtcacccgctcgattcctgagatagttgaggcggtaaaggcctccctgaagccctctaatgtcatagtcgagggcgagctggttgccgtcggcgagaacggtcgcccgaggcccttccagtacgtcctgaggcgctttaggaggaagtacaacatcgaggagatgattgagaagattccgctcgagctcaacctcttcgacatcctctacgttgaaggggagagcctcattgacacgaagttcgccgagaggaggaggaagcttgaggagagcgtcgaggagggcgataagataaagctcgccgaacagctcgttacgaagaaggtcgaagaggccgaggagttctacaagagggcccttgagctcggccacgagggcctgatggcgaagaggctggactccatctacgagcccggaaaccgcggtaagaagtggctgaagattaagcccacgatggagaaccttgacctcgtcattatcggagccgaatggggcgaggggaggcgcgcgcacctgcttggctcgttcctcgttgcggcctacgaccccgagagcggtgagttcgtcccggtgggcaaggtcgggagcggtttcaccgatgaagatttggtcgagttcaccaagatgctcaagcccctgattgtccgtgaagagggcaagttcgtcgagattgagcccaaggtcgtcatcgaggtcacctaccaggagatacagaagagccccaagtataagagcggtttcgcgcttcgcttcccgcgctacgtggcgctgagggaagataaaagcccggaggaggctgacaccatagagagggtcgcccagctctacgagctccaggagaggttcaaggcgaagaagtaatctaga

SEQ ID NO: 4 - DNA, 1695 bp, unknown
  Thermococcus sp. strain 9 degrees North
atgggcgatatgaggtactccgagctggccgaactctaccggaggcttgagaagaccacgctcaaaacgctcaagaccaagttcgtcgcggacttcctcaagaaaacacccgacgatttgctcgagatagttccctacctgattctcggcaaggtctttccggactgggacgagcgcgagctcggcgtcggggaaaagttgctcataagggccgtttcgatggcgactggggttccagagagggaaatcgagaactcgattaaggacacgggcgatttgggcgagagcgtggccttggccctaaagaagaggaagcagaagagcttcttcagccagcccctcacgataaagcgcgtctacagcaccttcgttaaggttgccgaggcgagcggagaggggagccaggacaggaagatgaagtacttagcaaacctcttcatggatgctcaacccgaggagggcaagtacatagccagaaccgtcctcgggacgatgaggacgggcgtcgccgagggaatcctgcgcgatgccatagcggaggccttcaaggtgaagccagagctcgtcgagagggcctacatgctcacgagcgacttcggctacgtggcgaaggttgcaaagctcgaggggaacgaggggctctcaaaggttagcatacagattgggaagccgataaggccgatgctagctcaaaacgccgccaacgtcaaggaagcgctaatcgagatgggcggtgaggcggccttcgagattaagtacgacggcgcgcgcgttcaggtccaccgcgatggggacagggtgataatctactcgaggaggcttgagaacgtcacccgctcgattcctgagatagttgaggcggtaaaggcctccctgaagccctctaatgtcatagtcgagggcgagctggttgccgtcggcgagaacggtcgcccgaggcccttccagtacgtcctgaggcgctttaggaggaagtacaacatcgaggagatgattgagaagattccgctcgagctcaacctcttcgacatcctctacgttgaaggagagagcctcatcgacacgaagttcgcagagaggaggaagaagctcgaggagagcgttgaggagagcgataagataaagctcgccgaacagctcgttacgaagaaggtcgaagaggccgaggagttctacaagagggcccttgagctcggccacgagggcctgatggcgaagaggctggactccatctacgagcccggaaaccgcggtaagaagtggctgaagattaagcccacgatggagaaccttgacctcgtcattatcggagccgaatggggcgaggggaggcgcgcgcacctgctcggctcgttcctcgttgcggcctacgaccccgagagcggtgagttcgtcccggtgggcaaggtcgggagcggtttcaccgatgaagatttggtcgagttcaccaagatgctcaagcccctgattgtccgtgaagagggcaagtttgtcgagattgagcccaaggtcgtcatcgaggtcacctaccaggagatacagaagagccccaagtataagagcggtttcgcgcttcgcttcccgcgctacgtggcgctgagggaagataaaagcccggaggaggccgacaccatagagagggtcgcccagctctacgagctccaggagaggttcaaggcgaagaagtaatctaga

SEQ ID NO: 5 - DNA, 1695 bp, unknown
  Thermoccocus sp. strain 9 degrees North
atgggcgatatgaggtactccgagctggccgaactctaccggaggcttgagaagaccacgctcaaaacgctcaagaccaagttcgtcgcggacttcctcaagaaaacacccgacgatttgctcgagatagttccctacctgattctcggcaaggtctttccggactgggacgagcgcgagctcggcgtcggggaaaagttgctcataagggccgtttcgatggcgactggggttccagagagggaaatcgagaactcgattaaggacacgggcgatttgggcgagagcgtggccttggccctaaagaagaggaagcagaagagcttcttcagccagcccctcacgataaagcgcgtctacagcaccttcgttaaggttgccgaggcgagcggagaggggagccaggacaggaagatgaagtacttagcaaacctcttcatggatgctcaacccgaggagggcaagtacatagccagaaccgtcctcgggacgatgaggacgggcgtcgccgagggaatcctgcgcgatgccatagcggaggccttcaaggtgaagccagagctcgtcgagagggcctacatgctcacgagcgacttcggctacgtggcgaaggttgcaaagctcgaggggaacgaggggctctcaaaggttagcatacagattgggaagccgataaggccgatgctagctcaaaacgccgccaacgtcaaggaagcgctaatcgagatgggcggtgaggcggccttcgagattaagtacgacggcgcgcgcgttcaggtccaccgcgacggggatagggtgataatatactcgaggaggcttgagaacgtcacccgctcgattcctgagatagtcgaggcggtaaaggcctccctgaagccttctaaggtcatagtcgagggcgagctggttgccgtcggcgagaacggtcgcccgaggcccttccagtacgtcctcaggaggttcaggaggaagtacaacatcgaggagatgattgagaagattccgctcgagctcaacctcttcgatatcctctacgttgatggggagagtctcatcgacacgaagttcgcagagaggaggaagaaactcgaggagagtgttgaggagagcgataagataaagctcgccgaacagctcgttacgaagaaggtcgaagaggccgaggagttctacaagagggcccttgagctcggccacgagggcctgatggcgaagaggctggactccatctacgagcccggaaaccgcggtaagaagtggctgaagattaagcccacgatggagaaccttgacctcgtcattatcggagccgaatggggcgaggggaggcgcgcgcacctgcttggctcgttcctcgttgcggcctacgaccccgagagcggtgagttcgtcccggtgggcaaggtcgggagcggtttcaccgatgaagatttggtcgagttcaccaagatgctcaagcccctgattgtccgtgaagagggcaagtttgtcgagattgagcccaaggtcgtcatcgaagtaacgtatcaggagatacagaagagccccaagtataagagcggtttcgcgcttcgcttcccgcgctacgtggcgctgagggaagataaaagcccggaggaggctgacaccatagagagggtcgcccagctctacgagctccaggagaggttcaaggcgaagaagtaatctaga

SEQ ID NO: 6 - DNA, 1695 bp, unknown
  Thermococcus sp. strain 9 degrees North
atgggcgacatgaggtactccgagctggccgaactctaccggaggcttgagaagaccacgctcaaaacgctcaagaccaagttcgtcgcggacttcctcaagaaaacacccgacgatttgctcgagatagttccctacctgattctcggcaaggtctttccggactgggacgagcgcgagctcggcgtcggggaaaagttgctcataagggccgtttcgatggcgactggggttccagagagggaaatcgagaactcgattaaggacacgggcgatttgggcgagagcgtggccttggccctaaagaagaggaagcagaagagcttcttcagccagcccctcacgataaagcgcgtctacagcaccttcgttaaggttgccgaggcgagcggagaggggagccaggacaggaagatgaagtacttagcaaacctcttcatggatgctcaacccgaggagggcaagtacatagccagaaccgtcctcgggacgatgaggacgggcgtcgccgagggaatcctgcgcgatgccatagcggaggccttcaaggtgaagccagagctcgtcgagagggcctacatgctcacgagcgacttcggctacgtggcgaaggttgcaaagctcgaggggaacgaggggctctcaaaggttagcatacagattgggaagccgataaggccgatgctagctcaaaacgccgccaacgtcaaggaagcgctaatcgagatgggcggtgaggcggccttcgagattaagtacgacggcgcacgcgttcaggtccaccgcgacggggacagggtgataatctactcgaggaggcttgagaacatcacccgctcgattcctgagatagtcgaggcggtaaaggcctccctgaagccttctaaggtcatagtcgagggcgagctggttgccgtcggcgagaacggtcgcccgaggcccttccagtacgtcctcaggaggttcaggaggaagtacaacatcgaggagatgattgagaagattccgctcgagctcaacctcttcgatatcctctacgttgatggggagagtctcatcgacacgaagttcgcagagaggaggaagaaactcgaggagagtgttgaggagagcgataagataaagctcgccgaacagctcgttacgaagaaggtcgaagaggccgaggagttctacaagagggcccttgagctcggccacgagggcctgatggcgaagaggctggactccatctacgagcccggaaaccgcggtaagaagtggctgaagattaagcccacgatggagaaccttgacctcgtcattatcggagccgaatggggcgaggggaggcgcgcgcacctgcttggctcgttcctcgttgcggcctacgaccccgagagcggtgagttcgtcccggtgggcaaggtcgggagcggtttcaccgatgaagatttggtcgagttcaccaagatgctcaagcccctgattgtccgtgaagagggcaagtttgtcgagattgagcccaaggtcgtcatcgaagtaacgtatcaggagatacagaagagccccaagtataagagcggtttcgcgcttcgcttcccgcgctacgtggcgctgagggaagataaaagcccggaggaggctgacaccatagagagggtcgcccagctctacgagctccaggagaggtacaaggcgaagaggtaatctaga

SEQ ID NO: 7 - DNA, 1695 bp, unknown
  Thermococcus sp. strain 9 degrees North
atgggcgatatgaggtactccgagctggccgaactttacagaaggcttgagaagaccacgctcaaaacgctcaagaccaagttcgtcgcggacttcctcaagaaaactcccgacgatttgctcgagatagttccctacctgattctcggcaaggtctttccggactgggacgagcgcgagctcggcgtcggggaaaagttgctcataagggccgtttcgatggcaaccggcgtccccgagagggaaatcgagaactcgattaaggacacgggcgatttgggcgagagcgtggctctggctctaaagaagaggaagcagaagagcttcttcagccagcccctcacgataaagcgcgtctacagcaccttcgttaaggttgccgaggcgagcggagaggggagccaggacaggaagatgaagtacttagcaaatctcttcatggatgctcaacccgaggagggcaagtacatagccagaaccgtcctcggaacgatgaggacgggcgtcgccgagggaatcctgcgcgatgccatagcggaggccttcaaggtgaagccagagctcgtcgagagggcctacatgctcacgagcgacttcggatacgtggcaaaggttgcaaagctcgaggggaacgaggggctctcaaaggttagcatacagattgggaagccgataaggccgatgctagctcaaaacgccgccaacgtcaaggaagcgctaatcgagatgggcggtgaggcggccttcgagattaagtacgacggtgcgcgcgttcaggtccaccgcgacggggatagggtgataatctactcgaggaggctcgagaacgtcacccgctcgattcctgagatagtcgaggcggtaaaggcctccctgaagccttctaaggtcatagtcgagggcgagctggttgccgtcggcgagaacggtcgcccgaggcccttccagtacgtcctcaggaggttcaggaggaagtacaacatcgaggagatgattgagaagattccgctcgagctcaacctcttcgatatcctctacgttgatggggagagtctcatcgacacgaagttcgcagagaggaggaagaaactcgaggagagtgttgaggagagcgataagataaagctcgccgaacagctcgttacgaagaaggtcgaagaggccgaggagttctacaagagggcccttgagctcggccacgagggcctgatggcgaagaggctggactccatctacgagcccggaaaccgcggtaagaagtggctgaagattaagcccacgatggagaaccttgacctcgtcattatcggagccgaatggggcgaggggaggcgcgcgcacctgcttggctcgttcctcgttgcggcctacgaccccgagagcggtgagttcgtcccggtgggcaaggtcgggagcggtttcaccgatgaagatttggtcgagttcaccaagatgctcaagcccctgattgtccgtgaagagggcaagttcgtcgagattgagcccaaggtcgtcatcgaggtcacctaccaggagatacagaagagccccaagtataagagcggtttcgcgcttcgcttcccgcgctacgtggcgctgagggaagataaaagcccggaggaggctgacaccatagagagggtcgcccagctctacgagctccaggagaggttcaaggcgaagaagtaatctaga

SEQ ID NO: 8 - protein, 564 aa, unknown
  Thermoccus sp. strain 9 degrees North
MGDMRYSELAELYRRLEKTTLKTLKTKFVADFLKKTPDDLLEIVPYLILGKVFPDWDERELGVGEKLLIRAVSMATGVPEREIENSIKDTGDLGESVALALKKRKQKSFFSQPLTIKRVYSTFVKVAEASGEGSQDRKMKYLANLFMDAQPEEGKYIARTVLGTMRTGVAEGILRDAIAEAFKVKPELVERAYMLTSDFGYVAKVAKLEGNEGLSKVSIQIGKPIRPMLAQNAANVKEALIEMGGEAAFEIKYDGARVQVHRDGDRVIIYSRRLENVTRSIPEIVEAVKASLKPSKVIVEGELVAVGENGRPRPFQYVLRRFRRKYNIEEMIEKIPLELNLFDILYVDGESLIDTKFAERRKKLEESVEESDKIKLAEQLVTKKVEEAEEFYKRALELGHEGLMAKRLDSIYEPGNRGKKWLKIKPTMENLDLVIIGAEWGEGRRAHLLGSFLVAAYDPESGEFVPVGKVGSGFTDEDLVEFTKMLKPLIVREEGKFVEIEPKVVIEVTYQEIQKSPKYKSGFALRFPRYVALREDKSPEEADTIERVAQLYELQERFKAKKSR

SEQ ID NO: 9 - protein, 564 aa, unknown; identical to SEQ ID NO: 8
  Thermococcus sp. strain 9 degrees North

SEQ ID NO: 10 - protein, 564 aa, unknown
  Thermococcus sp. strain 9 degrees North
MGDMRYSELAELYRRLEKTTLKTLKTKFVADFLKKTPDDLLEIVPYLILGKVFPDWDERELGVGEKLLIRAVSMATGVPEREIENSIKDTGDLGESVALALKKRKQKSFFSQPLTIKRVYSTFVKVAEASGEGSQDRKMKYLANLFMDAQPEEGKYIARTVLGTMRTGVAEGILRDAIAEAFKVKPELVERAYMLTSDFGYVAKVAKLEGNEGLSKVSIQIGKPIRPMLAQNAANVKEALIEMGGEAAFEIKYDGARVQVHRDGDRVIIYSRRLENVTRSIPEIVEAVKASLKPSNVIVEGELVAVGENGRPRPFQYVLRRFRRKYNIEEMIEKIPLELNLFDILYVEGESLIDTKFAERRKKLEESVEESDKIKLAEQLVTKKVEEAEEFYKRALELGHEGLMAKRLDSIYEPGNRGKKWLKIKPTMENLDLVIIGAEWGEGRRAHLLGSFLVAAYDPESGEFVPVGKVGSGFTDEDLVEFTKMLKPLIVREEGKFVEIEPKVVIEVTYQEIQKSPKYKSGFALRFPRYVALREDKSPEEADTIERVAQLYELQERFKAKKSR

SEQ ID NO: 11 - protein, 563 aa, unknown
  Thermococcus sp. strain 9 degrees North
MGDMRYSELAELYRRLEKTTLKTLKTKFVADFLKKTPDDLLEIVPYLILGKVFPDWDERELGVGEKLLIRAVSMATGVPEREIENSIKDTGDLGESVALALKKRKQKSFFSQPLTIKRVYSTFVKVAEASGEGSQRKMKYLANLFMDAQPEEGKYIARTVLGTMRTGVAEGILRDAIAEAFKVKPELVERAYMLTSDFGYVAKVAKLEGNEGLSKVSIQIGKPIRPMLAQNAANVKEALIEMGGEAAFEIKYDGARVQVHRDGDRVIIYSRRLENVTRSIPEIVEAVKASLKPSNVIVEGELVAVGENGRPRPFQYVLRRFRRKYNIEEMIEKIPLELNLFDILYVEGESLIDTKFAERRRKLEESVEEGDKIKLAEQLVTKKVEEAEEFYKRALELGHEGLMAKRLDSIYEPGNRGKKWLKIKPTMENLDLVIIGAEWGEGRRAHLLGSFLVAAYDPESGEFVPVGKVGSGFTDEDLVEFTKMLKPLIVREEGKFVEIEPKVVIEVTYQEIQKSPKYKSGFALRFPRYVALREDKSPEEADTIERVAQLYELQERFKAKKSR

SEQ ID NO: 12 - protein, 564 aa, unknown
  Thermoccocus sp. strain 9 degrees North
MGDMRYSELAELYRRLEKTTLKTLKTKFVADFLKKTPDDLLEIVPYLILGKVFPDWDERELGVGEKLLIRAVSMATGVPEREIENSIKDTGDLGESVALALKKRKQKSFFSQPLTIKRVYSTFVKVAEASGEGSQDRKMKYLANLFMDAQPEEGKYIARTVLGTMRTGVAEGILRDAIAESFKVKPELVERAYMLTSDFGYVAKVAKLEGNEGLSKVSIQIGKPIRPMLAQNAANVKEALIEMGGEAAFEIKYDGARVQVHRDGDRVIIYSRRLENVTRSIPEIVEAVKASLKPSNVIVEGELVAVGENGRPRPFQYVLRRFRRKYNIEEMIEKIPLELNLFDILYVEGESLIDTKFAERRKKLEESVEESDKIKLAEQLVTKKVEEAEEFYKRALELGHEGLMAKRLDSIYEPGNRGKKWLKIKPTMENLDLVIIGAEWGEGRRAHLLGSFLVAAYDPESGEFVPVGKVGSGFTDEDLVEFTKMLKPLIVREEGKFVEIEPKVVIEVTYQEIQKSPKYKSGFALRFPRYVALREDKSPEEADTIERVAQLYELQERFKAKRSR

SEQ ID NO: 13 - protein, 564 aa, unknown
  Thermococcus sp. strain 9 degrees North
MGDMRYSELAELYRRLEKTTLKTLKTKFVADFLKKTPDDLLEIVPYLILGKVFPDWDERELGVGEKLLIRAVSMATGVPEREIENSIKDTGDLGESVALALKKRKQKSFFSQPLTIKRVYSTFVKVAEASGEGSQDRKMKYLANLFMDAQPEEGKYIARTVLGTMRTGVAEGILRDAIAESFKVKPELVERAYMLTSDFGYVAKVAKLEGNEGLSKVSIQIGKPIRPMLAQNAANVKEALIEMGGEAAFEIKYDGARVQVHRDGDRVIIYSRRLENVTRSIPEIVEAVKASLKPSNVIVEGELVAVGENGRPRPFQYVLRRFRRKYNIEEMIEKIPLELNLFDILYVEGESLIDTKFAERRKKLEESVEESDKIKLAEQLVTKKVEEAEEFYKRALELGHEGLMAKRLDSIYEPGNRGKKWLKIKPTMENLDLVIIGAEWGEGRRAHLLGSFLVAAYDPESGEFVPVGKVGSGFTDEDLVEFTKMLKPLIVREEGKFVEIEPKVVIEVTYQEIQKSPKYKSGFALRFPRYVALREDKSPEEADTIERVAQLYELQERFKAKKSR

SEQ ID NO: 14 - protein, 564 aa, unknown
  Thermococcus sp. strain 9 degrees North
MGDMRYSELAELYRRLEKTTLKTLKTKFVADFLKKTPDDLLEIVPYLILGKVFPDWDERELGVGEKLLIRAVSMATGVPEREIENSIKDTGDLGESVALALKKRKQKSFFSQPLTIKRVYSTFVKVAEASGEGSQDRKMKYLANLFMDAQPEEGKYIARTVLGTMRTGVAEGILRDAIAEAFKVKPELVERAYMLTSDFGYVAKVAKLEGNEGLSKVSIQIGKPIRPMLAQNAANVKEALIEMGGEAAFEIKYDGARVQVHRDGDRVIIYSRRLENITRSIPEIVEAVKASLKPSKVIVEGELVAVGENGRPRPFQYVLRRFRRKYNIEEMIEKIPLELNLFDILYVDGESLIDTKFAERRKKLEESVEESDKIKLAEQLVTKKVEEAEEFYKRALELGHEGLMAKRLDSIYEPGNRGKKWLKIKPTMENLDLVIIGAEWGEGRRAHLLGSFLVAAYDPESGEFVPVGKVGSGFTDEDLVEFTKMLKPLIVREEGKFVEIEPKVVIEVTYQEIQKSPKYKSGFALRFPRYVALREDKSPEEADTIERVAQLYELQERYKAKRSR

SEQ ID NO: 15 - protein, 564 aa, unknown; identical to SEQ ID NO: 13
  Thermococcus sp. 9 degrees NB2

SEQ ID NO: 16 - protein, 559 aa, unknown
  Thermococcus sp. tfumicolans
MKYSELAGLYRRLEKTTLKTLKTRFVADFLKNVPDELLEIVPYLILGKVFPDWDERELGVGEKLLIKAVSIATGVPEGEIENSIKDTGDLGESIALAVKKKKQKSFFSQPLTIKRVYDTFVKVAESQGEGSQDRKMKYLANLFMDAQPEEAKYIARTVLGTMRTGVAEGILRDAIAEAFKVKAELVERAYMLTSDFGYVTKVAKLEGNEGLSKVRIQVGKPVRPMLAQNAASVKDALLEMGGEAAFEIKYDGARVQVHKDGDRVVIYSRRLENVTRSIPEIVEAVRSQLRPEKAIVEGELVAVGDGGKPRPFQYVLRRFRRKYNIEEMIERIPLELNLFDVLYVDGESLVDTPFMERRKRLEEAVEESERIKLAQQLVTKKAEEAEEFYRRALELGHEGLMAKRLDSVYEPGNRGKKWLKIKPTMEDLDLVIIGAEWGEGRRAHLLGSFLVAAYDQHRGEFVPVGKVGSGFTDEDLAEFTKMLKPLIVREEGKYVEIEPRVVIQVTYQEIQKSPKYESGFALRFPRYVALREDKSPEEADTIERISELYGLQERFKAKR

SEQ ID NO: 17 - protein, 562 aa, unknown
  Thermococcus sp. tkodakaraenis
MSDMRYSELADLYRRLEKTTLKTLKTKFVADFLKKTPDELLEIVPYLILGKVFPDWDERELGVGEKLLIKAVSMATGVPEKEIEDSVRDTGDLGESVALAIKKKKQKSFFSQPLTIKRVYDTFVKIAEAQGEGSQDRKMKYLANLFMDAEPEEGKYLARTVLGTMRTGVAEGILRDAIAEAFRVKPELVERAYMLTSDFGYVAKIAKLEGNEGLSKVRIQIGKPIRPMLAQNAASVKDALIEMGGEAAFEIKYDGARVQVHKDGDKVIVYSRRLENVTRSIPEVIEAIKAALKPEKAIVEGELVAVGENGRPRPFQYVLRRFRRKYNIDEMIEKIPLELNLFDVMFVDGESLIETKFIDRRNKLEEIVKESEKIKLAEQLITKKVEEAEAFYRRALELGHEGLMAKRLDSIYEPGNRGKKWLKIKPTMENLDLVIIGAEWGEGRRAHLLGSFLVAAYDPHSGEFLPVGKVGSGFTDEDLVEFTKMLKPYIVRQEGKFVEIEPKFVIEVTYQEIQKSPKYKSGFALRFPRYVALREDKSPEEADTIERVAELYELQERFKAKK

SEQ ID NO: 18 - protein, 559 aa, unknown
  Pyrococcus sp. pabyssi
MRYIELAQLYQKLEKTTMKLIKTRLVADFLKKVPEDHLEFIPYLILGDVFPEWDERELGVGEKLLIKAVSMATGIDSKEIENSVKDTGDLGESIALAVKRRKQKSFFSQPLTIKRVYQTLVKVAETTGEGSQDKKMKYLANLFMDAEPIEAKYIARTVLGTMRTGVAEGLLRDAISLAFNVKVELVERAYMLTSDFGFVAKIAKTEGNDGLAKVTIQIGKPIKPMLAQQAANIKEALLEMGGEAEFEIKYDGARVQVHKDGEKVTIYSRRLENVTRAIPEIVEAIKEALKPAKAIVEGELVAIGEDGRPLPFQYVLRRFRRKYNIEEMMEKIPLELNLFDVLYVDGVSLIDTKFMERRKKLEEIVEANGKVKIAENLITKNVEEAEQFYKRALEMGHEGLMAKRLDAIYEPGNRGKKWLKIKPTMENLDLVIIGAEWGEGRRAHLLGSFILGAYDPETGEFLEVGKVGSGFTDDDLVEFTKMLRPLIIKEEGKRVWIQPKVVIEVTYQEIQKSPKYRSGFALRFPRYVALREDKGPEDADTIERIAQLYELQERMKGKV

SEQ ID NO: 19 - protein, 561 aa, unknown
  Pyrococcus sp. pfuriosus
MRYLELAQLYQKLEKTTMKLIKTRLVADFLKKVPDDHLEFIPYLILGEVFPEWDERELGVGEKLLIKAVAMATGIDAKEIEESVKDTGDLGESIALAVKKKKQKSFFSQPLTIKRVYQTLVKVAETTGEGSQDKKVKYLADLFMDAEPLEAKYLARTILGTMRTGVAEGLLRDAIAMAFHVKVELVERAYMLTSDFGYVAKIAKLEGNEGLAKVQVQLGKPIKPMLAQQAASIRDALLEMGGEAEFEIKYDGARVQVHKDGSKIIVYSRRLENVTRAIPEIVEALKEAIIPEKAIVEGELVAIGENGRPLPFQYVLRRFRRKHNIEEMMEKIPLELNLFDVLYVDGQSLIDTKFIDRRRTLEEIIKQNEKIKVAENLITKKVEEAEAFYKRALEMGHEGLMAKRLDAVYEPGNRGKKWLKIKPTMENLDLVIIGAEWGEGRRAHLFGSFILGAYDPETGEFLEVGKVGSGFTDDDLVEFTKMLKPLIIKEEGKRVWLQPKVVIEVTYQEIQKSPKYRSGFALRFPRFVALRDDKGPEDADTIERIAQLYELQEKMKGKVES

SEQ ID NO: 20 - DNA, 31 bp, unknown
  primer
cggtggtgcatatgrgcgayatgmrstactc

SEQ ID NO: 21 - DNA, 40 bp, unknown
  primer
ataaactctagattacytcttcgccttgaacctctcctgg

SEQ ID NO: 22 - DNA, 39 bp, unknown
  primer
cggtggtgcatatgggcgayatgaggtactccgagctgg

SEQ ID NO: 23 - DNA, 23 bp, unknown
  primer
cgaacgtcgcgcagagaaacagg

SEQ ID NO: 24 - DNA, 20 bp, unknown
  primer
cctgctctgccgcttcacgc

(19) United States
(12) Patent Application Publication
(10) Pub. No.: US 2009/0142811 A1
(43) Pub. Date: Jun. 4, 2009

(54) Discovery, Cloning and Purification of Thermococccus sp. (Strain 9 Degrees N-7) Dna Ligase

(75) Inventors: Ira Schildkraut, Cerrillos, NM (US); Ezra Schildkraut, Cerrillos, NM (US)

Correspondence Address: HARRIET M. STRIMPEL, D. Phil., New England Biolabs, Inc., 240 COUNTY ROAD, IPSWICH, MA, 01938-2723, US

(73) Assignee: New England Biolabs, Inc., Ipswich, MA (US)

(21) Appl. No.: 12/067,000

(22) PCT Filed: Sep. 15, 2006
(86) PCT No.: PCT/US06/35919

§ 371 Date: Mar. 14, 2008

Related U.S. Application Data
(60) Provisional application No. 60/717,296, filed on Sep. 15, 2005.

(51) Int. Cl. C12P 19/34; C12N 9/00; C07H 21/04; C12N 15/74

(52) U.S. Cl. 435/91.52; 435/183; 536/23.2; 435/320.1

(57) ABSTRACT

Compositions that describe a thermostable DNA ligase isolated from Thermococcus sp. (strain 9° N-7) and methods for making und using the same are described. The thermostable DMA ligase depends on ATP and not NAD+ as a cofactor during ligation, and retains activity at 100° C.

### BACKGROUND

Thermococcus is a genus of the phylum Archaea. These ancient organisms grow in diverse environments under extreme conditions including high temperatures. The ability to grow these organisms in the laboratory is very limited so that little is known about their biochemistry or their metabolism.

Ligases are enzymes that catalyze the formation of a phosphodiester bond at the site of a single-stranded break in duplex DNA. The ligase enzyme also catalyzes the covalent linkage of duplex DNA generally blunt end to blunt end, or one cohesive end to another cohesive end. Ligases have been cloned from a variety of bacteria including one heat stable ligase-Thermus aquaticus (Taq ligase). This ligase has been described in U.S. Pat. No. 6,054,564.

Only a few Thermococci have been isolated and little is known about the properties of ligases they may contain or the genes encoding such proteins (see for example, Nakatani et al. J. Bacteriology 182: 6424-6433 (2000)). A ligase from a different genus of Archaea—a Pyrococcus, has been described in, for example, U.S. Pat. Nos. 5,506,137 and 5,700,672 and in Keppetipola et al. J. Bacteriology 187:6902-6908 (2005).

Ligases are used in many techniques in molecular biology including DNA amplification, sequencing and detection of single nucleotide polymorphisms. There is a continued need to find improved ligases that are stable at high temperatures and have rapid kinetics and stringent specificity.

### SUMMARY

In an embodiment of the invention, a substantially pure recombinant protein having DNA ligase activity is provided where the protein has at least 91% amino acid sequence identity with SEQ ID NO:13.

In a further embodiment of the invention, a substantially pure protein having DNA ligase activity is provided where the DNA ligase is encoded by a DNA sequence selected from the group consisting of: (a) a sequence that substantially the same as SEQ ID NO:2; (b) a sequence that is substantially complementary to SEQ ID NO:2, (c) a sequence that is substantially hybridizes to SEQ ID NO:2 under stringent conditions; and (d) a sequence encoding SEQ ID NO:13.

The protein referred to in the above embodiments is further characterized in that at least 25% of ligase activity is retained by the ligase after 30 minutes incubation at a temperature of about 100° C. Moreover, the ligase may be further characterized by its use of ATP as a cofactor during ligation whereas NAD⁺ provides no detectable utility for this purpose.

In a further embodiment, a DNA encoding a DNA ligase is provided having a sequence selected from the group consisting of: (a) a sequence that is substantially the same as SEQ ID NO:2; (b) a sequence that is substantially complementary to SEQ ID NO:2, (c) a sequence that hybridizes to SEQ ID NO:2 under stringent conditions; and (d) a sequence encoding SEQ ID NO:13.

In a further embodiment, a vector is described that contains the DNA described above. In addition, a host cell is provided that is capable of expressing the ligase from the vector.

In a further embodiment, a method of ligating a phosphodiester bond is provided that includes: selecting a DNA ligase of the type described above; mixing the ligase with a DNA, the DNA containing a break in at least one strand of the DNA; and ligating the phosphodiester bond at the break.

In an example of the method, the DNA ligase is a thermostable ligase from an archaeal isolate more particularly Thermococcus sp. (strain 9° N-7).

### BRIEF DESCRIPTION OF THE DRAWINGS

FIGS. 1a-1-1a-5 show DNA sequence alignments of 9° N-7 DNA ligase variants (SEQ ID NOS:1-7).

FIGS. 1b-1-1b-2 show protein alignments of Thermococcus sp. (strain 9° N-7) DNA ligase variants (SEQ ID NOS:8-15).

FIG. 2 shows a plasmid map of Thermococcus sp. (strain 9° N-7) DNA ligase gene inserted into litmus 28i.

FIG. 3 shows a plasmid map of Thermococcus sp. (strain 9° N-7) DNA ligase gene inserted into pMalC2x.

FIG. 4 shows protein alignments of Thermococcus sp. (strain 9° N-7) (SEQ ID NO:15) with Thermococcus fumicolans (SEQ ID NO:16), Thermococcus kodakaraensis (SEQ ID NO:17), Pyrococcus abyssi (SEQ ID NO:18), and Pyrococcus furiosus (SEQ ID NO:19).

FIG. 5 shows an SDS PAGE of phosphocellulose column fractions. Lanes are labeled as follows:

FT (flow-through of column) refers to fraction numbers 23-34;

MW refers to molecular weight standards.

The arrow indicates the position of a band corresponding to DNA ligase on the gel.

FIG. 6 shows thermostability of 9° N-7 DNA ligase. 30 μl of 10 mM Tris HCl pH 7.5, 2.5 mM MgCl₂, 2.5 mM DTT, 300 μM ATP and 0.1% Triton X-100 containing 3 μl of a 1:100 dilution of purified 9° N-7 DNA ligase was further diluted serially 3 fold in 10 mM Tris HCl pH 7.5, 2.5 mM MgCl₂, 2.5 mM DTT, 300 μM ATP and 0.1% Triton X-100. Four identical sets of dilutions were incubated for 30 minutes at 4° C., 80° C., 90° C. or 100° C.

To terminate the reaction, the samples were placed on ice and an equal volume of 10 mM Tris HCl, pH 7.5, 2.5 mM MgCl₂, 2.5 mM DTT, 300° C. μM ATP, 0.1% Triton X-100 and 50 μg/ml of BstEII Lambda DNA was added to each tube. The reactions were then incubated at 45° C. for 15 minutes after which a 0.15 volume of 50% glycerol, 100 mM EDTA and bromophenol blue was added to each tube. The reactions were then incubated at 75° C. for 5 minutes and electrophoresed on 1% agarose TBE gel.

Panel A shows the results of incubation on ice for 30 minutes.

Panel B shows the results of incubation at 80° C. for 30 minutes.

Panel C shows the results of incubation at 90° C. for 30 minutes.

Panel D shows the results of incubation at 100° C. for 30 minutes.

For each panel the lanes are designated as follows:

Lane 1 represents no further dilution;

Lane 2 was diluted 1:3;

Lane 3 was diluted 1:9;

Lane 4 was diluted 1:27; and

Lane 5 was diluted 1:81.

FIG. 7 shows a gel in which 9° N-7 polymerase was compared with Taq polymerase in a repair mixture containing E. coli polymerase and E. coli Endo IV. The repair mixture was incubated with depurinated DNA and amplified.

Lane 1 is a control;

Lane 2 is the DNA in the absence of a repair mixture;

Lanes 3 and 4 are duplicate samples of DNA and a repair mix containing 480 units Taq ligase; and

Lanes 5 and 6 are duplicate samples of DNA and a repair mix containing 500 units of 9° N-7 ligase.

### DETAILED DESCRIPTION OF THE EMBODIMENTS

The term “thermostable ligase” is used here to refer to an enzyme that catalyzes ligation of DNA and retains at least 25% of its activity after 30 minutes at 100° C. This thermostability under extreme temperatures is a characteristic that distinguishes the Thermococcus ligase (archaea) from Thermus ligase (bacteria).

Thermococcus sp. (strain 9° N-7) is a Thermococcus species isolated from hydrothermal vents (Southworth et al. PNAS 93:5281 (1996)).

The two known closest relatives to Thermococcus sp. (strain 9° N-7) DNA ligase are Thermococcus fumicolans ligase and Thermococcus kodakaraensis ligase (JP 2000308494-A/1), which share respectively 88% and 90% identity on the amino acid level. Both of these are reported to utilize either NAD⁺ or ATP as cofactors thereby constituting a new class of ligase. While T. fumicolans ligase is reported by Nakatani et al. (J. Bacteriology 182:6424-6433 (2000)) to utilize either NAD⁺ or ATP equally well and T. kodakaraensis ligase was active at a reduced level using NAD⁺ instead of ATP, Thermococcus sp. (strain 9° N-7) had no detectable activity with NAD⁺ (Example 2).

The terms “substantially the same” and “substantially complementary” are intended to mean that the DNA or amino acid sequence is largely the same or identical to the identified sequence or is largely the same or identical to the complementary sequence. The terms are intended to encompass sequences containing minor differences in amino acid or DNA sequence to that specified in the Figures. Such differences may arise from mutagenic events that do not significantly interfere with the ligation function of the protein.

In an embodiment of the invention, stringent hybridization is conducted under the following conditions: a) hybridization: 0.75M NaCl, 0.15 Tris HCl, 10 mM EDTA, 0.1% NaCl, 0.1% SLS, 0.03% BSA, 0.03% Ficoll 400, 0.03% PVP and 100 μg/ml boiled calf thymus DNA at 50° C. for about 12 hours and; b) wash 3 times for 30 minutes with 0.1×SET, 0.1% SDS, 0.1% NaCl and 0.1M phosphate buffer at 45° C. and the presence of double-stranded hybridized DNA detected on a Southern Blot.

All references cited herein as well as U.S. provisional application 60/717,296 filed Sep. 15, 2005 are incorporated by reference.

### EXAMPLES

### Example I

### Cloning Thermococcus sp. (Strain 9° N-7) DNA Ligase Gene Using Degenerate Primers

The gene was first amplified from Thermococcus sp. (strain 9° N-7) genomic DNA by PCR. Sequences for forward primers were derived from the references by Nakatani et al. J. Bact. 182(22):6424-6433 (2000) (Thermococcus kodakaraensis) and Rolland et al. FEMS Microbiology Lett. 236(2):267-273 (2004) (Thermococcus fumicolans). Consensus sequences with designated degeneracy were designed as follows:

(SEQ ID NO: 20)

Forward Primer
5′CGGTGGTGCATATGRGCGAYATGMRSTACTC

(SEQ ID NO: 21)

Reverse Primer
5′ATAAACTCTAGATTACYTCTTCGCCTTGAACCTCTCCTGG

The primers for Thermococcus sp. (strain 9° N-7) were used to amplify the gene for DNA ligase from genomic Thermococcus sp. (strain 9° N-7) DNA. The PCR reaction conditions used to clone the gene were as follows:

100 μl reaction mix containing 20 mM Tris-HCL, pH 8.8, 10 mM KCl, 10 mM (NH₄)₂SO₄ with 4 mM MgSO₄, 0.1% Triton X-100, 200 μM each dNTP, 50 ng of Thermococcus sp. (strain 9° N-7) genomic DNA, 500 ng each of forward and reverse primer, 2.5 units of Taq DNA polymerase and 0.02 units of Vent® DNA polymerase was heated to 94° C. for 1 minute, then brought to 45° C. for 1 minute and then brought to 72° C. for 3 minutes. The temperature cycle was repeated 30 times. After cycling was completed the reaction temperature was reduced to room temperature and 5 units of E. coli DNA polymerase Klenow fragment was added and incubated for a further 5 minutes at room temperature. The reaction was then adjusted to 70 mM EDTA. The PCR product was phenol extracted, alcohol precipitated and desalted on CL6B sepharose spin column.

The 1700 bp PCR product was cloned into E. coli. EcoRV-cleaved litmus 28i was used as the vector to clone the DNA fragment.

A 10 μl ligation reaction in T4 DNA ligase buffer contained 80 ng of the insert, 80 ng of litmus vector and 400 units of T4 DNA ligase (New England Biolabs, Inc., Ipswich, Mass.). The ligation reaction was incubated at 16° C. overnight, electroporated into E. coli TB1 cells and plated on IPTG XGAL plates.

The white colonies were picked. One out of nine white colonies had a 1700 bp insert. An independent electroporation yielded another clone with 1700 bp insert. The inserts in these two clones were sequenced.

From the sequence of these clones, a new less degenerate forward primer was designed as follows:

(SEQ ID NO: 22)

9°N forward primer:
5′cggtggtgcatatgggcgayatgaggtactccgagctgg

(2) Cloning Thermococcus sp. (strain 9° N-7) Ligase Using a Second Forward Primer that was Less Degenerate then the Primer in (1)

Four additional independent PCR reactions were performed using the 9° N-7 forward primer, which contained only one degenerate base in place of the forward primer in (1) above, which contained 5 degenerate bases.

100 μl of Phusion HF buffer (New England Biolabs, Inc., Ipswich, Mass.) containing 50 ng of Thermococcus sp. (strain 9° N-7) genomic DNA, 500 ng each of forward and reverse primer, 200 μM each dNTP and 1 μl Phusion DNA polymerase (New England Biolabs, Inc., Ipswich, Mass.) was heated to 98° C. for 30 seconds and then 25 cycles of 98° C. for 10 seconds, followed by 70° C. for 30 seconds followed by 72° C. for 1 minute. The reaction was then incubated 72° C. for 5 minutes. The product of each of the PCR reactions was treated as the initial PCR reaction and cloned into litmus 28i as described above. Two independent clones from the PCR reaction (A1 and A3) were confirmed by miniprep DNA to contain a 1700 base pair inserts as well as one clone from each of the other three PCR reactions (B2, C3, D3). These clones were then grown and their crude extracts were electrophoresed on SDS PAGE. Each of the clones expressed a 60 kd protein.

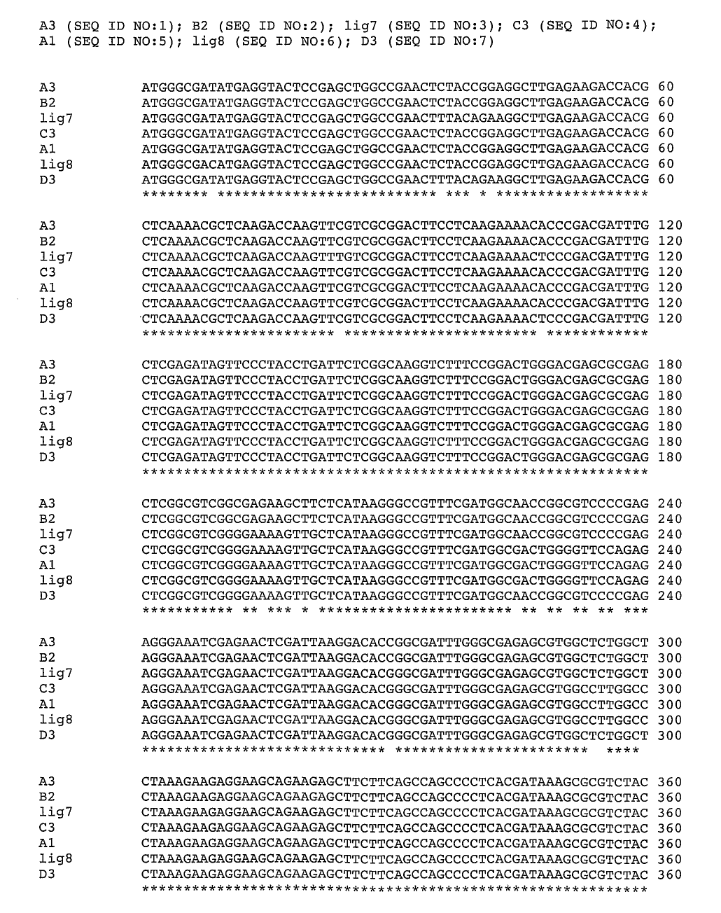

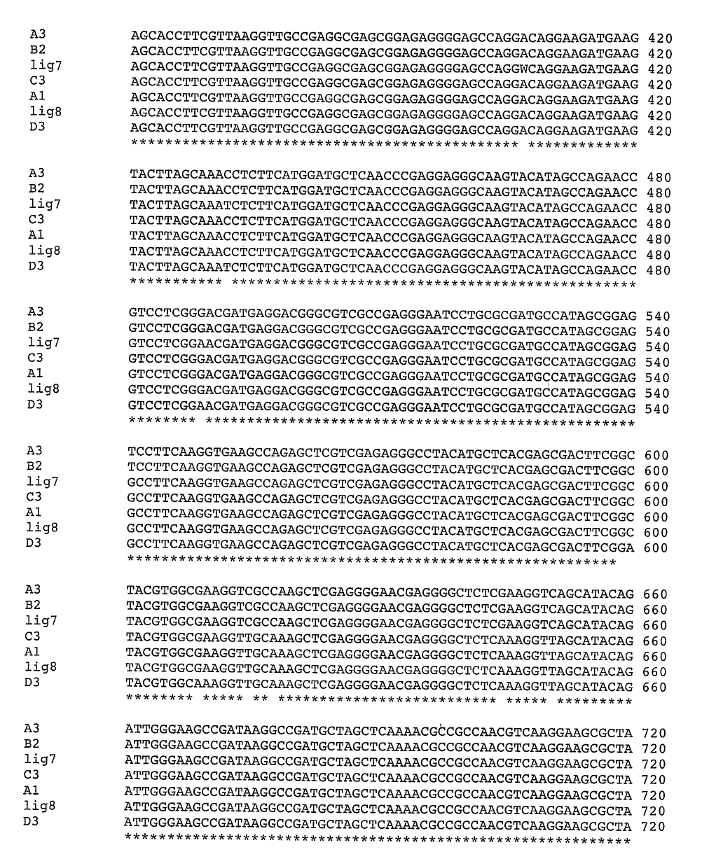

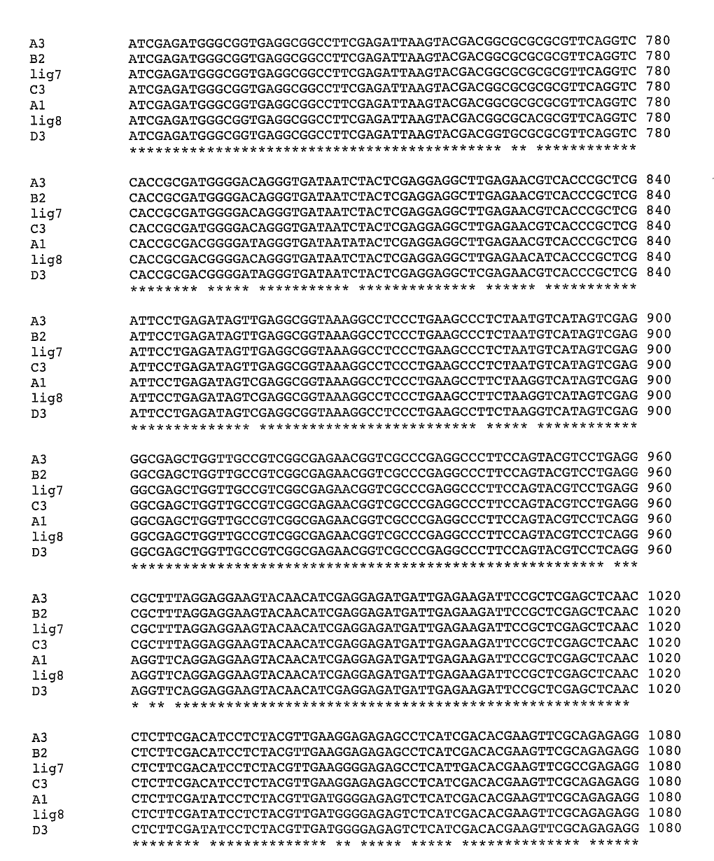

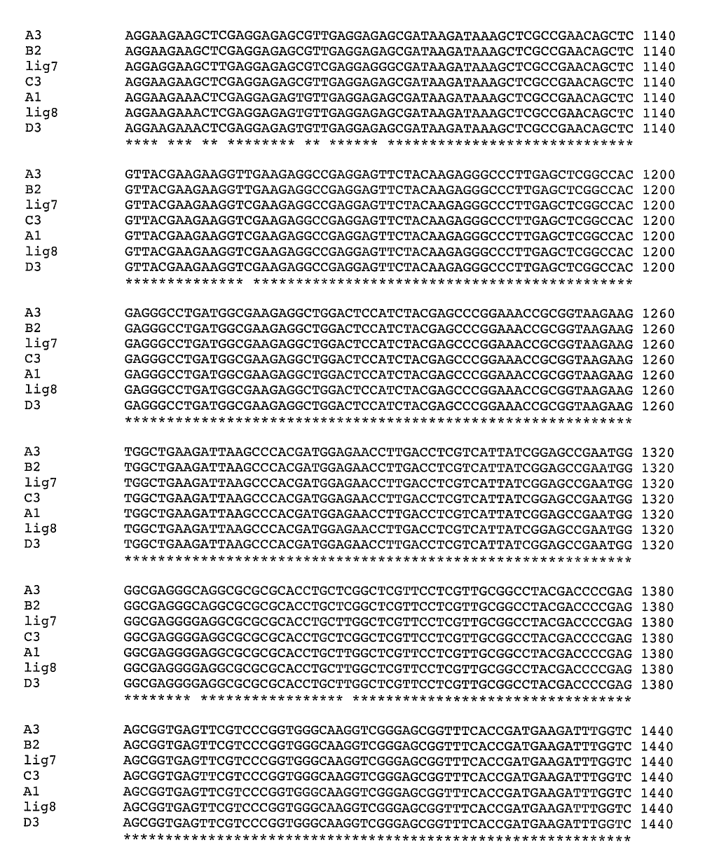

Plasmids from clones A1, A3, B2, C3, D3 and additionally lig7 and lig8 were purified and the inserts sequenced. The DNA sequences are provided in FIGS. 1a-1-1a-5 (SEQ ID NOS:1-7).

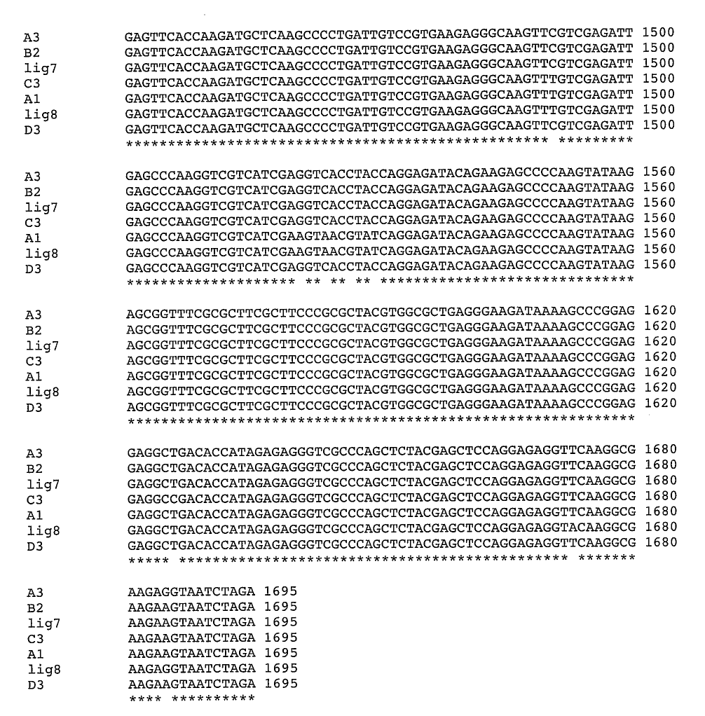

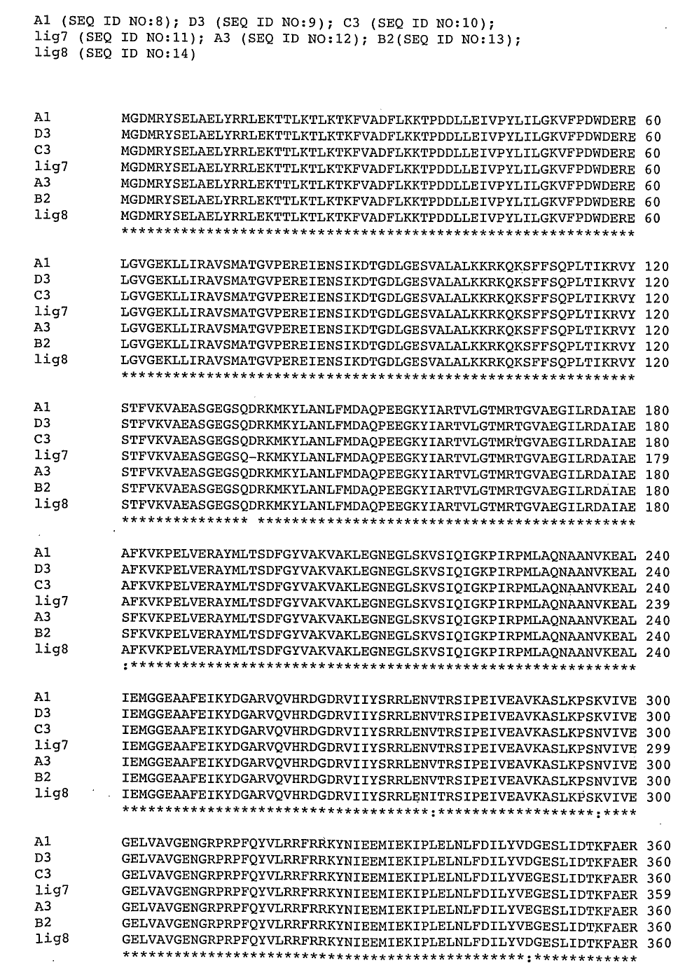

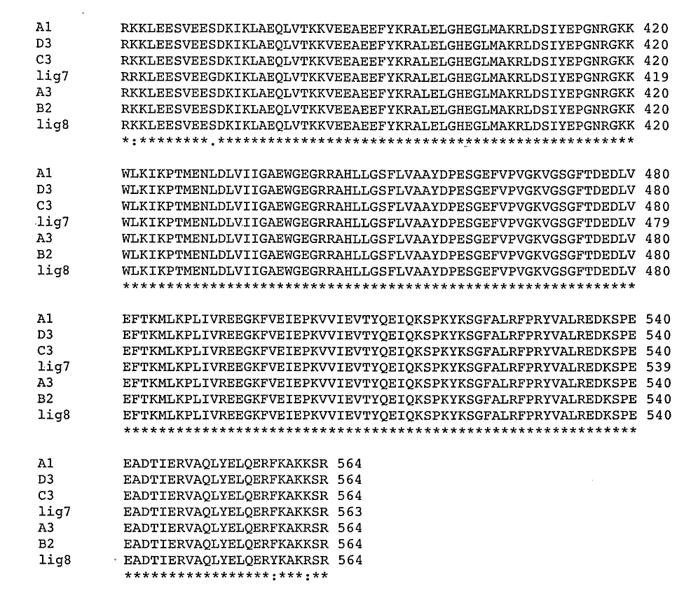

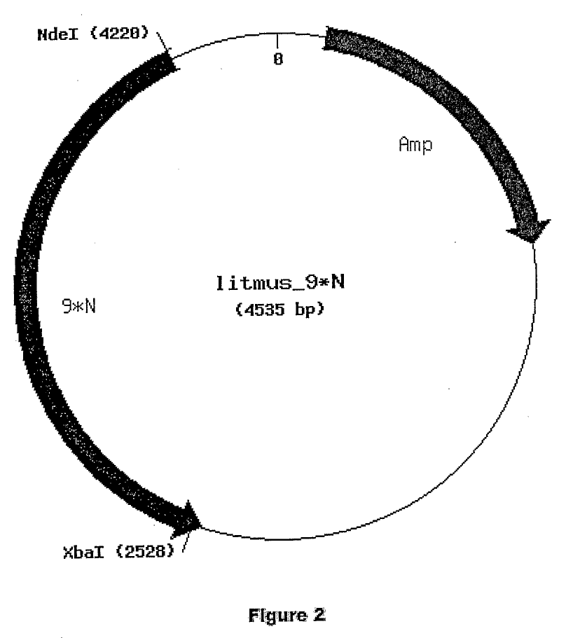

While not wishing to be limited by theory, the observed minor differences in sequences may be accounted for by clonal variation within the population of Thermococcus sp. (strain 9° N-7) cells. The sequence variations are all third position changes or conserved amino acid changes. Clone B2 is representative of the consensus sequence of the ligase. The DNA ligase was first expressed in a tightly controlled expression vector (FIG. 2).

(3) Expressing the Ligase Gene (B2) in E. coli

The B2 fragment was excised from the litmus vector by cleavage with NdeI and XbaI. The 1700 bp fragment was cut from the agarose gel and the gel slice was digested with agarase to release the fragment.

The expression vector pMalC2X (New England Biolabs, Inc., Ipswich, Mass.) was prepared by cleaving with NdeI and XbaI and dephosphorylated. The 1700 base pair cleaved PCR fragment was ligated to the pMalC2X vector in a 10 μl reaction containing 400 ng of insert and 100 ng of vector in T4 DNA ligase buffer and 200 units of T4 DNA ligase incubated at 16° C. for 16 hours. The ligation reaction was electroporated into E. coli TB1 cells and a clone carrying the 1700 bp fragment was isolated and designated Thermococcus sp. (strain 9° N-7) B2-1 (FIG. 3).

The clone was grown in LB media and induced with IPTG. A sample of the induced cells was lysed and electrophoresed in a SDS PAGE gel to reveal a band corresponding to a protein of size at ˜60 kd. The analysis of the protein sequence derived from the DNA sequence indicated the gene encoded a protein with 26 rare arginine codons. Therefore host cells containing the rare tRNA for arginine (E. coli BL-2 (DE3) RIL) (Stratagene, La Jolla, Calif.) were used to obtain higher levels of expression. After induction of the Thermococcus sp. (strain 9° N-7), B2-1 plasmid in the host sample was analyzed by SDS PAGE and a significant 60 kd band was observed.

### (4) Comparison of Thermococcus sp. (Strain 9° N-7) Ligase With Other Thermostable DNA Ligases

Thermococcus sp. (strain 9° N-7) DNA ligase amino acid sequence was compared by CLUSTAL multiple sequence alignment to 4 other thermophilic DNA ligases.

### CLUSTAL W (1.82) Multiple Sequence Alignments

Sequence format is Pearson.

### Sequence 1: 9° N-7-B2 (SEQ ID NO:15) 564 aa

Sequence 2: T. kodakaraenis (SEQ ID NO:16) 562 aa
Sequence 3: P. abyssi (SEQ ID NO:17) 559 aa
Sequence 4: P. furiosus (SEQ ID NO:18) 561 aa
Sequence 5: T. fumicolans (SEQ ID NO:19) 559 aa

### Identity Scores:

Sequences (1:2) Aligned. Score: 90
Sequences (1:3) Aligned. Score: 81
Sequences (1:4) Aligned. Score: 78
Sequences (1:5) Aligned. Score: 88
Sequences (2:3) Aligned. Score: 80
Sequences (2:4) Aligned. Score: 80
Sequences (2:5) Aligned. Score: 87
Sequences (3:4) Aligned. Score: 90
Sequences (3:5) Aligned. Score: 78
Sequences (4:5) Aligned. Score: 77

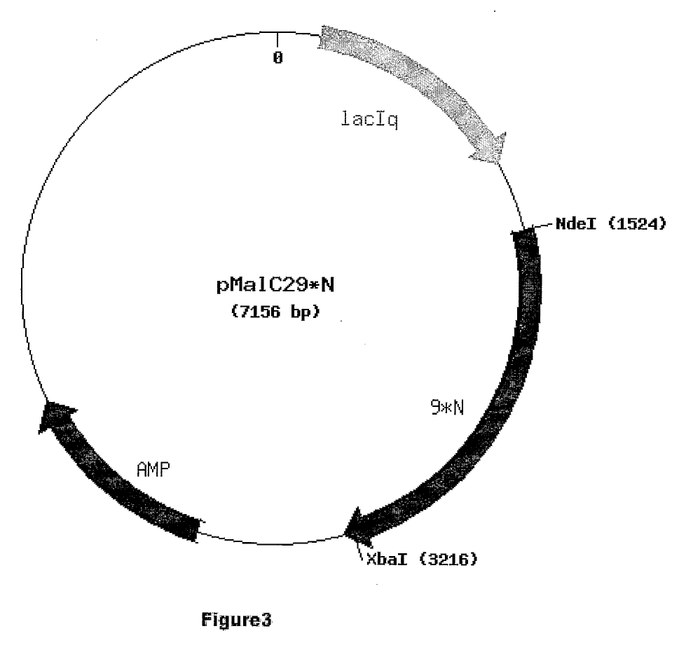

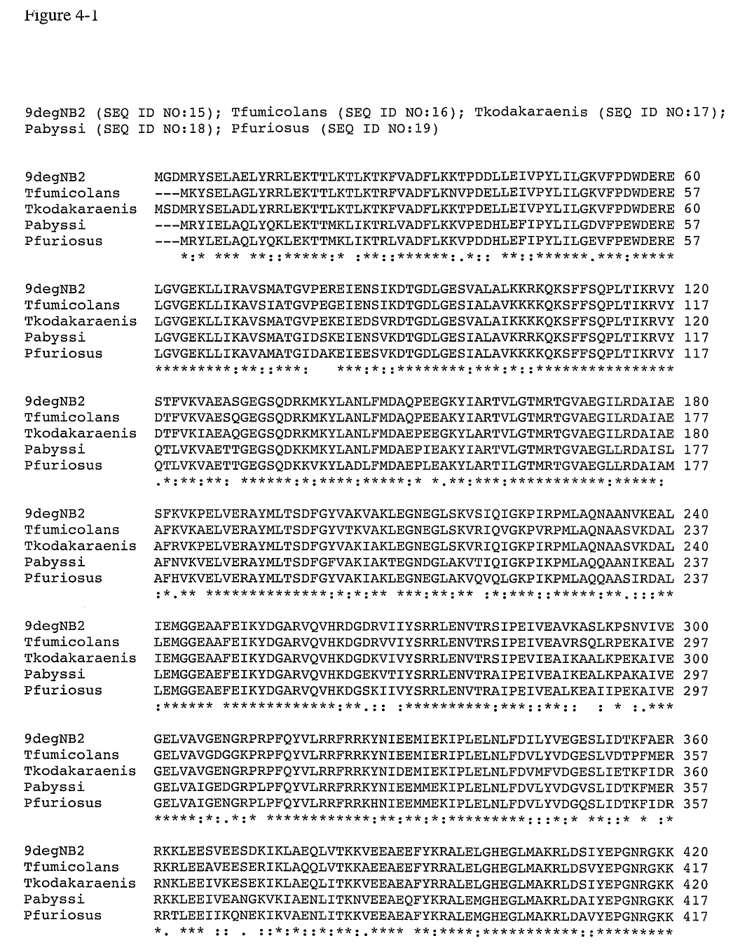

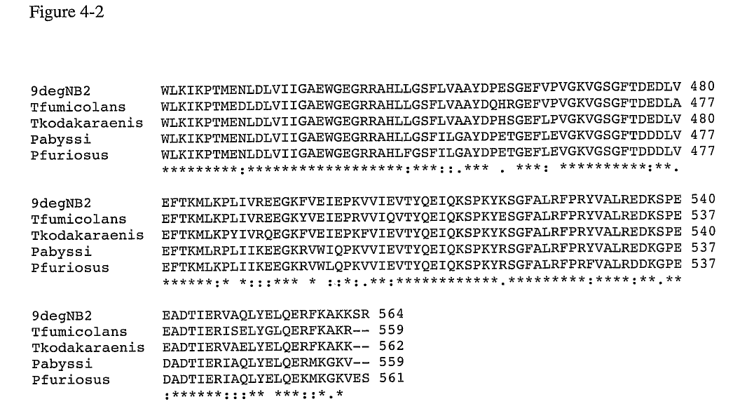

The alignments are presented in FIG. 4. The closest known relative to Thermococcus sp. (strain 9° N-7) DNA ligase is that of Thermococcus kodakaensis DNA ligase where there is 90% amino acid identity and 80.9% nucleotide identity.

### (5) Purification of Thermococcus sp. (Strain 9° N-7) DNA Ligase

E. coli BL-21 (DE3)-RIL (Stratagene, La Jolla, Calif.) was transformed with pMalC2X plasmid (New England Biolabs, Inc., Ipswich, Mass.) containing the B2 fragment for DNA ligase from Thermococcus sp. (strain 9° N-7). The cells were grown in 100 ml LB media with 50 μg/ml ampicillin and 25 μg/ml chloramphenicol at 37° C. After overnight incubation the culture was transferred to a ten-liter fermenter and incubated at 37° C. until an OD600 of 0.59 was achieved and that 0.1 gram of IPTG was added. The culture was incubated another 5.75 hours and harvested. The cell paste was stored at −20° C.

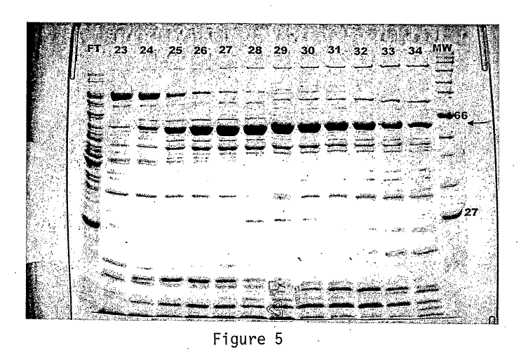

10 grams of cell paste in 40 ml of 10 mM Tris HCl, pH 7.5, 20 mM NaCl, 0.1 mM EDTA and 1.0 mM DTT were thawed and lysed by sonication. The extract was brought to 0.3 mM PMSF and 200 mM NaCl. The extract was clarified by centrifugation. The clarified extract was passed through a DEAE sepharose column at 0.2 M NaCl. The protein that flowed through the column was pooled and diluted to 100 mM NaCl. This was applied to a phosphocellulose column and the protein that was absorbed was eluted with a gradient of 100 mM to 1.1 M NaCl. The fractions (FIG. 5) were analyzed by SDS PAGE and the major 60 kd peak was pooled and heated to 75° C. for 30 minutes. This solution was clarified by centrifugation and the clarified solution was diluted to 100 mM NaCl and applied to a hydroxyapatite column. A 0-13 % gradient of ammonium sulfate was applied to the column and fractions collected and assayed for activity by incubating various fractions in T4 DNA ligase buffer (New England Biolabs, Inc., Ipswich, Mass.) with HindIII lambda DNA at 50 μg/ml as a substrate. The reactions were incubated at 37° C. for 10 minutes. The reaction was terminated by addition of 10% 100 mM EDTA and 50% glycerol and bromophenol blue dye. The reactions were heated to 650 and loaded onto 1% agarose gel for analysis.

The tubes containing about 80% of the ligase activity were pooled and dialyzed against 50% glycerol, 10 mM Tris HCl, pH 7.5, 50 mM KCl, 10 mM (NH₄)₂SO₄, 0.1 mM EDTA and 1.0 mM DTT. The purified Thermococcus sp. (strain 9° N) DNA ligase was stored at −20 C.

### Example 2

### Properties of Thermococcus sp. (Strain 9° N-7) DNA Ligase

The recommended reaction conditions are:

### 10 mM Tris-HCl pH 7.5

### 2.5 mM MgCl₂

### 2.5 mM DTT

### 300 uM ATP

The typical substrate for assaying activity at 45° C. is lambda DNA. Appropriately digested lambda DNA can reveal the state of ligation of the 12-base extension at the termini of lambda DNA. We typically used either a HindIII or BstEII predigested lambda DNA. Analysis of the ligation was performed on agarose gel electrophoresis.

The Km for ATP appears to be less than 100 μM. The activity was stimulated by Triton X-100.

Unlike Thermococcus fumicolans DNA ligase, Thermococcus sp. (strain 9° N-7) ligase in the presence of NAD⁺ had no detectable activity

The enzyme requires magnesium ions. 2.5 mM MgCl₂ achieved 10 times more activity than 10 mM MgCl₂.

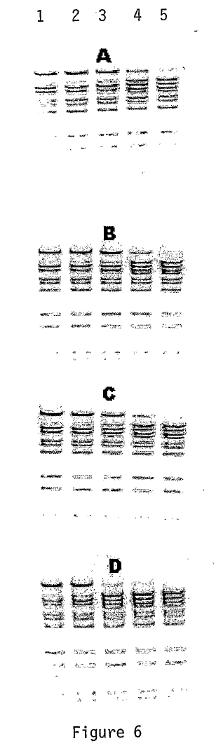

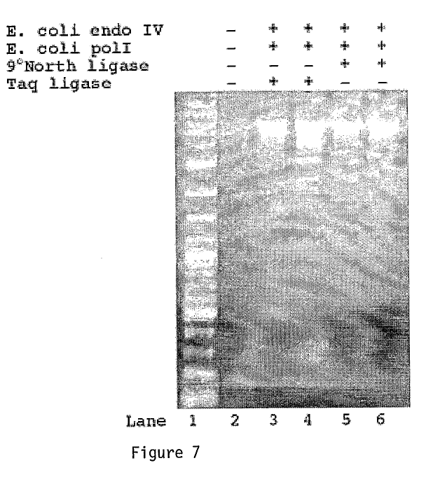

Between 25% and 50% of the activity remained after incubating the enzyme at about 100° C. for 30 min (FIG. 6).

The ligase is capable of sealing nicked DNA at 90° C. The DNA ligase was incubated with a BstNBI nicked pUC19 plasmid DNA and converted the relaxed nicked plasmid to the position of covalently closed circular DNA as determined by agarose gel electrophoresis. The rate of the reaction was higher at 80° than at 45° C. Although the nicked plasmid underwent denaturation at 90° C., substantial nick sealing occurred at 90° C. before denaturation converted all of the nicked plasmid to single strands.

### Example 3

### Use of Thermococcus sp. (Strain 9° N-7) DNA Ligase in a DNA Repair Mix

Repair of DNA damaged by depurination was achieved using a mixture of enzymes that included strain 9° N-7 DNA ligase.

The DNA in the experimental reaction was damaged by depurination as described by Ide, H., et al. Biochemistry 32(32):8276-83 (1993). Lambda DNA (NEB#N3011, New England Biolabs, Inc., Ipswich, Mass.) was ethanol precipitated. The DNA was resuspended in depurination buffer (100 mM NaCl, 10 mM citrate, pH 5.0) at a concentration of 0.5 mg/ml and incubated at 70° C. for 120 minutes. The sample was then ethanol precipitated and resuspended in a solution of 0.01 M Tris, 0.001 M EDTA, pH 8.0. The DNA concentration was determined by measuring the A²⁶⁰ of the DNA-containing solutions after calibrating with a buffer control.

The damaged DNA was incubated at room temperature in the following enzyme mixture for 10 minutes as follows: DNA (1 ng); 100 μM dNTPs (NEB#M0447, New England Biolabs, Ipswich, Mass.); 1 mM ATP; 480 units Taq ligase (NEB#M0208, New England Biolabs, Ipswich, Mass.) or 500 units of 9° N-7 DNA ligase (NEB#M0238, New England Biolabs, Ipswich, Mass.); 0.1 unit E. coli DNA polymerase I (E. coli polI) (NEB#M0209, New England Biolabs, Inc., Ipswich, Mass.); 10 units E. coli Endo IV (NEB#M0304, New England Biolabs, Inc., Ipswich, Mass.); 1×Thermopol buffer (NEB#B9004, New England Biolabs, Inc., Ipswich, Mass.) to a final volume of 47.5 μL.

At the end of the reaction, the samples were transferred to ice and then amplified. A negative control was treated as above, but without the enzymes.

### DNA Amplification Reaction

DNA amplification of lambda was performed using the following primers: CGAACGTCGCGCAGAGAAACAGG (L72-5R) (SEQ ID NO:23) and CCTGCTCTGCCGCTTCACGC (L30350F) (SEQ ID NO:24) according to the method of Wang et al. Nucl. Acids Res. 32:1197-1207(2004).

2.5 μl of amplification mixture was added to 47.5 ml of the above repair mixture. The amplification mixture contained 100 μM dNTPs, 5 units Taq DNA polymerase (New England Biolabs, Inc., Ipswich, Mass.), 0.1 unit Vent® (exo+) DNA polymerase (New England Biolabs, Inc., Ipswich, Mass.), 5×10⁻¹¹ M primer L72-5R and 5×10⁻¹¹ M primer L30350F in 1×Thermopol buffer.

To correct for any enzyme storage buffer effects, when a repair enzyme was omitted from a reaction, the appropriate volume of its storage buffer was added to the reaction. In all cases, the amplification reactions were processed in a thermal cycler using the following parameters: 20 seconds at 95° C. for 1 cycle followed by 5 seconds at 94° C., then 5 minutes at 72° C. for 25 cycles. The size of the amplicon being amplified was 5 kb.

The results of amplification of DNA (5 kb) were determined by 1% agarose gel elecrophoresis. 6×loading dye (Molecular Cloning: A Laboratory Manual, 3rd ed., eds. Sambrook and Russell, Cold Spring Harbor Press, Cold Spring Harbor, N.Y., 2001, pp. 5.4-5.17) was added to the 50 μl amplification reactions. 20 μl of this solution was then loaded onto the agarose gel along with 1 μg of 2-log ladder (NEB#N3200, New England Biolabs, Inc., Ipswich, Mass.) as a size standard.

What is claimed is:

1. A substantially pure recombinant protein having DNA ligase activity and having at least 91% amino acid sequence identity with SEQ ID NO:13.

2. A substantially pure protein having DNA ligase activity encoded by a DNA sequence selected from the group consisting of:
(a) a sequence substantially the same as SEQ ID NO:2;
(b) a sequence substantially complementary to SEQ ID NO:2,
(c) a sequence that hybridizes to SEQ ID NO:2 under stringent conditions; and
(d) a sequence encoding SEQ ID NO:13.

3. The protein according to claim 1, wherein at least 25% of ligase activity is retained after a 30 minute incubation at a temperature of about 100° C.

4. The protein according to claim 2, wherein at least 25% of ligase activity is retained after a 30 minute incubation at a temperature of about 100° C.

5. A protein according to claim 1, 2, 3 or 4 that can utilize ATP but not NAD⁺ as a cofactor during ligation.

6. A DNA encoding a DNA ligase, the DNA having a sequence selected from the group consisting of:
(a) a sequence substantially the same as SEQ ID NO:2;
(b) a sequence substantially complementary to SEQ ID NO:2,
(c) a sequence that hybridizes to SEQ ID NO:2 under stringent conditions; and
(d) a sequence encoding SEQ ID NO:13.

7. A vector containing the DNA of claim 6.

8. A host cell capable of expressing the protein of claim 1.

9. A method of ligating a phosphodiester bond, comprising:
(a) selecting a ligase according to claim 1 or 2;
(b) mixing the ligase with a DNA, the DNA containing a break in at least one strand of the DNA; and
(c) ligating the phosphodiester bond at the break.

10. A method according to claim 9, wherein the ligase is a thermostable ligase from an archaeal isolate.

11. A method according to claim 9, wherein the archaeal isolate is Thermococcus sp (strain 9° N-7).
